(19) United States
(12) Patent Application Publication
(10) Pub. No.: US 2002/0054894 A1
(43) Pub. Date: May 9, 2002

(54) Composition comprising a hydrophilic component and a combination of a volatile compound and a pasty fatty compound, and use thereof

(76) Inventors: Isabelle Agostini, Chatenay Malabry (FR); Dolores Miguel-Colombel, L'Hay-Les-Roses (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/951,446

(22) Filed: Sep. 14, 2001

Related U.S. Application Data
(63) Continuation of application No. 08/896,049, filed on Jul. 17, 1997.

(30) Foreign Application Priority Data
Jul. 17, 1996 (FR) 96-08957

(51) Int. Cl. A61K 6/00; A61K 7/00; A61K 7/021; A61K 7/025; A61K 7/035; A01N 25/00

(52) U.S. Cl. 424/401; 424/63; 424/64; 424/69; 424/70.1; 424/70.7; 514/844; 514/845; 514/846; 514/897; 514/944

(57) ABSTRACT

A cosmetic, dermatological, hygiene and/or pharmaceutical composition comprising a hydrophilic component and a fatty phase which comprises at least one volatile compound and at least one pasty fatty compound. The composition is, in particular, a composition having improved staying power. The use of the combination of a volatile compound and a pasty fatty compound to limit the transfer and/or migration of such a cosmetic, dermatological, hygiene and/or pharmaceutical composition comprising a hydrophilic compound.

The present invention relates to a composition, in particular a cosmetic composition, which may be in the form of a stick or a soft paste and which may be used to care for and/or make up the skin, semi-mucous membranes and/or mucous membranes, and in particular the lips of the face.

The solid, fatty cosmetic or pharmaceutical compositions usually used especially in the form of pencils, for example lipstick pencils, have the drawback of being relatively non-moisturizing. To overcome this drawback, it has been attempted to introduce water therein by preparing emulsified products. Mention may be made in this respect of French patent application FR 2,237,615, which describes a water-in-oil emulsion containing from 1 to 50% by weight of water. However, this type of composition poses problems of evaporation of the water. The composition described in that patent application also contains from 1 to 10% of a polyhydroxylated compound in order to obtain uniform distribution of the dye substances in the water-in-oil emulsion.

The problem of evaporation of the water from solid dispersions has been solved according to European patent application EP 374,332 by the incorporation of a silicone oil and a polyorganosiloxane modified with a polyoxyalkylene. The compositions thus modified may also contain polyols in a low amount as additional wetting agents.

Japanese patent application JP 01-143,812 describes the incorporation of a large amount of polyvalent alcohols into a solid emulsion for cosmetic use by means of a silicone oil and a denatured polyoxyalkylene polyorganosiloxane. However, it is difficult to keep the formulation homogeneous with that system.

Lastly, application EP 524,892 is known, the aim of which is to overcome the drawbacks described above by proposing a specific process which makes it possible to obtain a solid, anhydrous dispersion which comprises fatty substances and a polyhydric alcohol in a large amount.

However, it was observed that when these compositions are applied to the skin, mucous membranes or semi-mucous membranes, they have the drawback of transferring. This is understood to mean that the composition is liable to be deposited, at least partly, on certain supports with which it is placed in contact, such as, for example, a glass, clothing or the skin. On being deposited, the composition leaves a trace on the support. This results in mediocre persistence of the composition on the skin or the mucous membranes, making it necessary to repeat its application regularly.

Another drawback of these compositions lies in the problem of migration. This is because it has been seen that certain compositions have a tendency to spread inside the wrinkles and/or fine lines of the skin, in the case of foundations; in the fine lines around the lips, in the case of lipsticks; in the folds of the eyelid, in the case of eyeshadows.

Especially in the case of eyeshadows, the appearance of lines in the make-up which are generated by the movements of the eyelids has also been observed. All of these phenomena produce an unpleasant-looking make-up effect which it would obviously be desirable to avoid.

These drawbacks, which are generally present for common lipstick compositions, are even more pronounced when the composition comprises polyols. This is due in particular to the fact that polyols are sticky and viscous compounds which tend to adhere to and be deposited on the support with which they are placed in contact, leaving a trace thereon.

There is thus a need for a composition, in particular a cosmetic composition, which transfers little or not at all, that is to say a “non-transferring” composition, while at the same time possessing good moisturizing and/or emollient properties.

In particular, there is a need for a composition which is capable of comprising hydrophilic compounds such as polyols in a large amount, while at the same time retaining very good staying power, that is to say not transferring and not marking, or marking only very little, a support with which it might come into contact, and not migrating over time.

The aim of the present invention is thus to propose such a composition, which transfers little or not at all, although it comprises hydrophilic compounds.

The subject of the invention is thus a cosmetic, dermatological, hygiene and/or pharmaceutical composition comprising a hydrophilic component and a fatty phase which comprises at least one volatile compound and at least one pasty fatty compound.

The subject of the invention is also the use of the combination of a volatile compound and a pasty fatty compound in order to limit, decrease and/or eliminate transfer and/or migration, and/or in order to improve the staying power, of a cosmetic, dermatological, hygiene and/or pharmaceutical composition comprising a hydrophilic component.

The subject of the invention is also a process for limiting, decreasing and/or eliminating the transfer of a cosmetic, dermatological, hygiene and/or pharmaceutical composition comprising a hydrophilic component, in particular a make-up and/or care composition for the skin, mucous membranes, semi-mucous membranes and/or the exoskeleton, this process comprising introducing into the composition at least one volatile compound and at least one pasty fatty compound.

It has been observed that the composition according to the invention can be applied easily to the skin and that it affords, inter alia, a make-up of light texture which remains comfortable to wear throughout the day. Furthermore, the composition according to the invention possesses excellent emollient properties when applied to the skin.

It has moreover been observed that the composition according to the invention makes it possible to obtain a film of homogeneous coloration due to good moistening of the pigments in the pasty fatty compounds; the film can be applied easily and spreads easily on the support.

The composition according to the invention finds a particularly advantageous application in the field of caring for and/or making up the skin, mucous membranes, semi-mucous membranes and the exoskeleton. The term mucous membrane is understood in particular to refer to the inner part of the lower eyelid; the term semi-mucous membranes is understood to refer more particularly to the lips of the face; the term exoskeleton is understood to refer to the eyelashes, the eyebrows, the hair and the nails. Thus, the invention finds an application most particularly in the field of make-up products for the lips of the face and also lipcare products, as well as in the field of make-up and care products for the skin such as foundations, self-tanning agents or antisun products.

The composition according to the invention thus comprises at least one hydrophilic component, which may preferably be a polyhydric alcohol and/or optionally a thickened or even gelled, aqueous phase.

The polyhydric alcohol may be a compound having from 2 to 8 carbon-atoms and 2 to 6 hydroxyl functions, such as ethylene glycol, glycerol, 1,2-propanediol, diglycerol, erythritol, arabitol, adonitol, sorbitol and dulcitol. The polyhydric alcohol may also be a polyether alcohol with an average molecular weight of from 150 to 600, such as polyethylene glycol 300 and polyglycerol 500, wherein the molecular weight is weight-average molecular weight. A mixture of polyhydyric alcohols may also be used.

The thickened or gelled aqueous phase may comprise water and/or a flower water such as cornflower water, and/or a mineral water such as Eau de Vittel, Eau de Lucas or Eau de La Roche Posay.

It may be thickened by the presence of any thickener known to those skilled in the art. Mention may be made in particular of:

extracts of algae such as agar-agar, carrageenans and alginates,

seed extracts such as extracts of carob or of guar,

fruit extracts, in particular pectin,

plant exudates such as gum arabic, gum tragacanth, karaya gum and ghatti gum,

cellulose derivatives such as carboxy-methylcellulose,

gelling agents of animal origin such as gelatin or caseinates,

micro-organism exudates such as xanthan gum,

synthetic gelling agents such as acrylic polymer derivatives (CARBOMER, CARBOPOL, PEMULEN) or silicon derivatives (LAPONITE, LAPOMER or VEEGUM).

The thickener may be present in the aqueous phase in a proportion of 0.2 to 10% by weight relative to the aqueous phase.

In addition, the hydrophilic component may comprise water-soluble additives, in particular vitamins, cosmetic and/or pharmaceutical active agents, dyes, amino acids and UV screening agents.

The hydrophilic component may represent 4 to 40% by weight of the composition, preferably 8 to 25% by weight.

Preferably, the hydrophilic component is dispersed in at least a part of the fatty phase, before being mixed with the rest of the constituents of the composition. When the hydrophilic component comprises at least one polyhydric alcohol, the particle size of the alcohol is preferably less than or equal to 1 micron.

In order to obtain a finer dispersion of the hydrophilic component in the composition, at least one surfactant may be added to the composition. This surfactant may be anionic or nonionic, preferably with an HLB of less than 10, more preferably less than 5. Mention may be made in particular of silicone derivatives such as silicone surfactants, or any other common surfactant such as lecithins, succinylglycerides, alkyl phosphates, magnesium lanolate and mixtures thereof.

The composition according to the invention thus also comprises a fatty phase, which comprises in particular volatile compounds. These volatile compounds may be selected in particular from hydrocarbon oils and/or silicone oils, which are cyclic or linear, alone or as a mixture.

In the present description, the term volatile compound is understood to refer to any compound which can evaporate through contact with the skin. Preferably, volatile oils whose flash point is sufficiently high to allow them to be used in formulation, and sufficiently low to obtain the desired evanescent effect are used. Volatile oils whose flash point it about 40 to 100° C. are more preferably used.

Among the volatile silicone oils, mention may be made, alone or as a mixture, of

volatile cyclic silicones having from 3 to 8 and preferably from 4 to 6 silicon atoms. These are, for example, cyclotetradimethylsiloxane, cyclopenta-dimethylsiloxane or cyclohexadimethylsiloxane;

cyclocopolymers of the dimethylsiloxane/ methylalkylsiloxane type, such as Silicone FZ 3109 sold by the company Union Carbide, which is a dimethylsiloxane/methyloctylsiloxane cyclocopolymer;

volatile linear silicones having from 2 to 9 silicon atoms. These are, for example, hexamethyl-disiloxane or a PDMS of low viscosity (1 cSt). Mention may also be made of alkyltrisiloxanes such as hexylheptamethyltrisiloxane or octylheptamethyl-trisiloxane.

Among the volatile hydrocarbon oils, mention may be made of isoparaffins and in particular isododecane.

The composition according to the invention may comprise 8 to 70% by weight, preferably 15 to 55%, of volatile compounds relative to the total weight of the composition.

The composition according to the invention also comprises at least one pasty fatty compound. This compound preferably comprises at least one hydrocarbon compound; it may be in the form of a polymer; it may also contain silicone and/or fluorine; it may also be in the form of a mixture of various hydrocarbon compounds and/or silicone compounds and/or fluoro compounds. In the case of a mixture, the hydrocarbon compounds are preferably predominantly used.

The pasty fatty compounds according to the invention may be defined by means of at least one of the following physicochemical properties:

a viscosity of from 0.1 to 40 Pa.s (1 to 400 poises), preferably 0.5 to 25 Pa.s, measured at 40° C. with a Contraves TV rotary viscometer equipped with an MS-r3 or MS-r4 rotor at a frequency of 60 Hz,

a melting point of 25 to 70° C., preferably 25 to 55° C.

A person skilled in the art may choose the rotor which allows the viscosity to be measured, from the rotors MS-r3 and MS-r4, on the basis of his or her general knowledge, so as to be able to measure the viscosity for the test compound.

Among the pasty compounds which may be used within the context of the present invention, mention may be made of lanolins or lanolin derivatives having a viscosity of 18 to 21 Pa.s, preferably 19 to 20.5 Pa.s, and/or a melting point of 30 to 60° C.

Mention may also be made of fatty esters, in particular those having 20 to 45 carbon atoms (melting point of about 25 to 70° C.), as well as triglycerides such as hydrogenated plant oils. Among the esters, mention may be made of arachidyl propionate, polyvinyl laurate and cholesterol esters.

Mention may also be made of pasty silicone fatty substances such as alkyidimethicones, which have a melting point of 25 to 60° C., in particular those sold by Dow Corning under the trade names DC2503 and DC25514.

Any common oil thickened with a common thickener may also be used as pasty fatty compound.

The oils which may be thickened may be of mineral, plant, animal and/or synthetic origin such as a silicon oil, and optionally phenolated. One or more of the oils mentioned below may be used in particular.

The thickener may be selected from clays such as bentonites or hectorites, optionally modified in particular with distearyidimethylammonium chloride or with stearyidimethylbenzylammonium chloride, or with aluminium or magnesium silicates, or alternatively with common polymers known to be capable of thickening oils.

Hydrogenated castor oil derivatives such as “THIXINR” from Rheox may also be used.

The pasty compound or compounds may be present in a proportion of from 1 to 40% by weight, preferably in a proportion of 8 to 35% by weight and even more preferably in a proportion of 15 to 30% by weight, in the composition.

The composition according to the invention may also comprise at least one wax, which can in particular provide the composition with mechanical strength, when it is in the form of a stick. When it is in the form of a soft paste or a cast product, the composition according to the invention may comprise a lesser amount of wax, for example about 2 to 15% by weight relative to the weight of the composition.

Any wax known in the prior art may be used, among which, alone or as a mixture, mention may be made of animal, plant, mineral and synthetic waxes such as microcrystalline waxes, paraffin, petrolatum, petroleum jelly, ozokerite, montan wax, beeswax, lanolin and derivatives thereof, candelilla wax, ouricurry wax, carnauba wax, Japan wax, cocoa butter, cork fibre wax or sugar cane wax; hydrogenated oils which are solid at 25° C., fatty esters and glycerides which are solid at 25° C.; polyethylene waxes and the waxes obtained by Fischer-Tropsch synthesis; silicone waxes; fluoro waxes.

Preferably, the waxes forming part of the composition may have a melting point above about 45° C. and in particular above 55° C., and/or a needle penetration index at 25° C. preferably of from 3 to 40. The composition preferably comprises 0.5 to 30% by weight of wax, in particular 10 to 20% by weight.

Moreover, the composition may comprise, in addition to the compounds mentioned above, oils of plant, mineral, animal and/or synthetic origin, in particular silicone oils.

Mention may thus be made of liquid paraffin, liquid petroleum jelly, perhydrosqualene, groundnut oil, sweet almond oil, beauty-leaf oil, palm oil, castor oil, avocado oil, jojoba oil, olive oil or cereal germ oil; fatty acid esters; alcohols; acetylglycerides; octanoates, decanoates or ricinoleates of alcohols or of polyalcohols; fatty acid triglycerides; glycerides.

Mention may also be made of polydimethylsiloxanes (PDMS) and alkyldimethicones, as well as silicones modified with aliphatic and/or aromatic groups, which may optionally contain fluorine, or with functional groups such as hydroxyl, thiol and/or amine groups.

Mention may also be made of phenylated silicone oils, in particular those of the following formula:

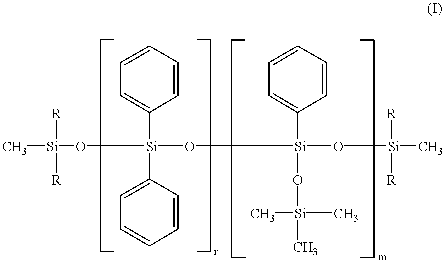

in which

R is a C₁-C₃₀ alkyl radical, an aryl radical or an aralkyl radical,

n is an integer from 0 to 100,

m is an integer from 0 to 100, with the proviso that the sum m+n is from 1 to 100.

Preferably, R is a methyl, ethyl, propyl, isopropyl, decyl, dodecyl or octadecyl radical or alternatively a phenyl, tolyl, benzyl or phenethyl radical.

Obviously, the fatty substances may be selected in particular in a varied manner by those skilled in the art in order to prepare a composition having the desired properties, for example in terms of consistency or texture.

In general, it is preferred to use the oils, which may be hydrocarbon and/or silicone oils, in a low amount, in particular less than 5% by weight relative to the total weight of the composition.

However, it has been observed that improvement in the staying power of the composition according to the invention, as well as its absence of migration and/or transfer, may be particularly advantageous when the composition comprises less than 20% by weight of non-volatile hydrocarbon oil, preferably less than 15% by weight, more preferably less than 5% by weight, or even no non-volatile hydrocarbon oils at all, which may thus be replaced by a larger amount of pasty fatty compounds.

In one particular embodiment of the invention, the composition may comprise only hydrocarbon fatty compounds, optionally without the volatile compounds. In particular, the composition may comprise lanolins and/or lanolin derivatives as pasty fatty compounds, hydrocarbon waxes and volatile compounds of isoparaffin or cyclic silicone type. This is because it has been observed that the moistening of the pigments is better when the fatty substances are hydrocarbons, which leads to a skin make-up of more homogeneous color. Moreover, the silicone fatty substances are not all compatible with all of the hydrocarbon fatty substances, which results in a greater limitation as regards the starting materials in the formulation.

It is also possible to add to the composition according to the invention an amount of a polymer which is sufficient to stabilize it further in a humid atmosphere. Such polymers should be liposoluble and possess a very low content of hydrophilic units. Among these polymers, mention may be made of polyalkylenes (in particular polyethylenes and polybutenes), polyacrylates and silicone polymers which are compatible with fatty substances. Among the polyalkylenes, mention may be made of polybutene, in particular that sold by the company Amoco under the name INDOPOL.

The weight ratio of the polymer to the polyhydric alcohol is preferably less than 4:1, more preferably from 0.25:1 to 2:1.

The composition may also comprise a particulate phase, which may comprise pigments and/or pearlescent agents and/or fillers usually used in cosmetic compositions. The term pigments should be understood to mean white or colored, inorganic or organic particles intended to color and/or opacify the composition. The term fillers should be understood to mean colorless or white, inorganic or synthetic, lamellar or non-lamellar particles intended to impart body or rigidity to the composition, and/or softness, a matt effect and uniformity to the make-up. The term pearlescent agents should be understood to mean iridescent particles which reflect light.

The pigments may be present in the composition in a proportion of from 0 to 15% by weight of the final composition, and preferably in a proportion of from 8 to 10% by weight. They may be white or colored, inorganic and/or organic, and of usual or nanometric size. Mention may be made of titanium dioxide, zirconium dioxide or cerium dioxide as well as zinc oxide, iron oxide or chromium oxide, ferric blue, carbon black, ultramarines (aluminosilicate polysulphides), manganese pyrophosphate and certain metal powders such as those of silver or of aluminium. Mention may also be made of the lakes commonly employed to impart a make-up effect to the lips and the skin, these lakes being calcium, barium, aluminium or zirconium salts of acidic dyes such as halo-acid, azo or anthraquinone dyes.

The pearlescent agents may be present in the composition in a proportion of from 0 to 20% by weight, preferably in a content of about 8 to 15% by weight. Among the pearlescent agents which may be envisaged, mention may be made of natural mother-of-pearl, mica coated with titanium oxide, with iron oxide, with natural pigment or with bismuth oxychloride, as well as colored titanium mica.

The fillers, which may be present in a proportion of from 0 to 30% by weight, preferably 5 to 15%, in the composition may be inorganic or synthetic, and lamellar or non-lamellar. Mention may be made of talc, mica, silica, kaolin, Nylon powders, polyethylene powders, Teflon, starch, boron nitride, microspheres such as EXPANCEL (Nobel Industrie), POLYTRAP (Dow Corning) and microbeads of silicone resin (TOSPEARLS from Toshiba for example).

The composition may also comprise any additive commonly used in the cosmetic field, such as antioxidants, fragrances, essential oils, preserving agents, lipophilic and/or hydrophilic cosmetic active agents, moisturizers, vitamins, essential fatty acids, sphingolipids, self-tanning agents such as DHA, and sunscreens.

These additives are found, depending on their solubility, either in the fatty phase or in the phase comprising the polyhydric alcohol.

Obviously, a person skilled in the art will take care to select the optional additional compounds, and/or the amount thereof, such that the advantageous properties of the composition according to the invention are not, or are not substantially, adversely affected by the addition envisaged.

The composition according to the invention may also comprise at least one active agent, among which mention may be made of agents that are active against microorganisms, in particular agents with antiviral, antibacterial or antifungal activity; agents with anti-inflammatory or immunomodulatory activity; agents which antagonize neuromediators or modulate the release of neuromediators; agents which modulate cell differentiation and/or proliferation and/or pigmentation and/or which regulate keratinization; agents that are active in the treatment and/or prevention of cheilitis; antihistamines; cicatrizing agents.

Depending on the pathologies to be treated, it may be prudent to incorporate several active agents, or alternatively to add to them secondary active agents which will enhance the prevention or treatment of the complaint, and/or to treat symptoms associated with this complaint. Among the secondary active agents, mention may be made of local anaesthetics, antiseptics, moisturizers and/or emollients, and sunscreens, in particular chemical or inorganic sunscreens.

The composition may also contain an agent for promoting the dissolution or compatibility of the active agents with the excipient of the composition, and/or for promoting the penetration of the active agents into the skin or mucous membranes. This agent may be, for example, isopropyl myristate, oleic acid, lecithin or certain alcohols.

The composition according to the invention may be in the form of a stick or pencil, in the form of an oily liquid, which may optionally be gelled, or alternatively in the form of a soft paste whose viscosity may be measured. The dynamic viscosity at 25° C. is generally from 3 to 35 Pa.s, measured with a Contraves TV rotary viscometer equipped with an “MS-r4” rotor at a frequency of 60 Hz.

The composition according to the invention may be prepared in the usual manner by those skilled in the art. In particular, the hydrophilic component may be incorporated into the composition by means of the use, during the incorporation, of a turbomixer at a minimum spin speed which produces particles of very small size, in particular smaller than or equal to 1 micron. A large amount of polyhydric alcohol, for example, may thus be incorporated.

In particular, the composition may be prepared by heating all or part of the fatty substances, except for the volatile compounds, and the hydrophilic component to a temperature of from 65 to 95° C., followed by mixing using a turbomixer at a spin speed of at least 1500 revolutions per minute, preferably 2500-3500 rev/min.

The volatile compounds and the optional additives liable to be heat-sensitive are then added. The emulsion thus obtained is poured into a suitable mold. The turbomixer which may be used in the process according to the invention may be of any model known commercially for the preparation of cosmetic or pharmaceutical compositions. Mention may be made, for example, of the Moritz turbomixer.

The composition according to the invention may also be prepared with the aid of one or more extruders, which makes it possible in particular to obtain a composition comprising a high content of waxes and being in the form of a soft paste.

The compositions according to the invention may be applied in particular to the field of making up the skin, semi-mucous membranes, mucous membranes and/or the exoskeleton, and are, in this case, for example, in the form of a foundation, a blusher, an eyeshadow, a lipstick, a mascara or an eyeliner.

The compositions may also be in non-colored form, optionally containing cosmetic active agents. In this case, they may be used in particular as a care base for the lips or as a fixing base to be applied to a standard lipstick. In this case, the fixing base forms a protective film over the film of lipstick, limits its transfer and migration and thus increases its staying power.

The compositions according to the invention may also be in the form of a care product for the skin, mucous membranes, semi-mucous membranes and/or the exoskeleton, a hygiene or pharmaceutical product or alternatively an antisun product or self-tanning product.

The invention is illustrated in greater detail in the examples which follow.

### EXAMPLE 1

A lipstick pencil having the following composition was prepared:

| | | |
|---|---|---|
| oxypropylenated lanolin (PPG5 lanolin wax; | 18 | g |
| viscosity 19 Pa.s) | | |
| triglycerides (hydrogenated cocoglycerides) | 6 | g |
| hydrocarbon waxes (in particular polyethylene waxes) | 17 | g |
| plant oil | 1.5 | g |
| pigments, pearlescent agents, fillers | 8 | g |
| glycerol | 10 | g |
| trioleyl phosphate | 1 | g |
| cyclotetradimethylsiloxane | qs 100 | g |

The composition was prepared in the usual manner, by heating the fatty substances, except for the volatile oils, to 95° C. and mixing them together. The pigments and the fillers were then added.

Separately, the glycerol was heated to 80° C.; a Moritz turbomixer was used at a spin speed of 300 rev/min to mix the fatty phase and the glycerol until a composition having a consistency which was capable of being poured was obtained; the volatile compounds were then added and the composition was poured into a suitable mold and left to cool.

A lipstick pencil with a pleasant texture, which spread well and applied uniformly was thus obtained. The film was comfortable to wear over time and did not migrate.

This composition was applied to the left part of the lips of several individuals.

For comparison, the same composition (control composition) in which the lanolin and the triglycerides are replaced by plant oils jojoba oil) was applied to the right part of the lips.

The lipsticks were left to dry at room temperature for 5 minutes and all of the lips were then pressed against a sheet of paper.

It was seen on all of the sheets of paper that a stronger trace of lipstick was left by the control composition. The composition according to the invention left a very faint, barely perceptible trace on the sheet.

### EXAMPLE 2

A lipstick was prepared in the form of a soft paste having the following

| | | |
|---|---|---|
| acetylated lanolin (viscosity 20.5 Pa.s) | 16 | g |
| triglycerides (hydrogenated cocoglycerides) | 6 | g |
| plant oil | 0.5 | g |
| hydrocarbon waxes (in particular polyethylene waxes) | 0.5 | g |
| pigments, pearlescent agents, fillers | 8 | g |
| trioleyl phosphate | 1 | g |
| glycerol | 10 | g |
| cyclopentadimethylsiloxane | qs 100 | g |

These various ingredients, except for the volatile oil, were introduced into a twin-screw extruder at an inlet temperature of about 75-95° C. The volatile oil was introduced into the extruder at the end of the extrusion, at a temperature of about 20-25° C. A soft paste was obtained at the outlet in the form of a single, stable, homogeneous phase which could be taken up with a brush in order to apply it.

This composition made it possible to obtain a homogeneous film which was easily applied and spread easily and uniformly. The film obtained also had a light texture and remained comfortable to wear throughout the day. It did not transfer and did not migrate.

### EXAMPLE 3

A lipstick fixing base was prepared in the form of a pencil having the following composition:

| | | |
|---|---|---|
| acetylate lanolin (viscosity 20.5 Pa.s) | 16 | g |
| triglycerides | 6 | g |
| plant oil | 0.5 | g |
| hydrocarbon waxes (in particular polyethylene waxes) | 17 | g |
| fillers | 0.5 | g |
| glycerol | 10 | g |
| trioleyl phosphate | 1 | g |
| isoparaffin | qs 100 | g |

The film obtained by applying the composition spread easily and uniformly onto a standard lipstick.

What is claimed is:

1. A cosmetic, dermatological, hygiene and/or pharmaceutical composition comprising
at least one hydrophilic component and
a fatty phase which comprises at least one volatile compound and at least one pasty fatty compound.

2. A non-transferring cosmetic, dermatological, hygiene and/or pharmaceutical composition comprising
at least one hydrophilic component and
a fatty phase which comprises at least one volatile compound and at least one pasty fatty compound.

3. A composition according to claim 1, wherein said at least one hydrophilic component is selected from a polyhydric alcohol, a thickened aqueous phase, and a gelled aqueous phase.

4. A composition according to claim 1, wherein said at least one hydrophilic component is selected from compounds having from 2 to 8 carbon atoms and from 2 to 6 hydroxyl functions and polyether alcohols with an average molecular weight of 150 to 600.

5. A composition according to claim 4, wherein compounds having from 2 to 8 carbon atoms and from 2 to 6 hydroxyl functions are selected from ethylene glycol, glycerol, 1,2-propanediol, diglycerol, erythritol, arabitol, adonitol, sorbitol and dulcitol.

6. A composition according to claim 4, wherein said polyether alcohols are selected from polyethylene glycol 300 and polyglycerol 500.

7. A composition according to claim 3, wherein said aqueous phase is thickened using at least one thickener selected from extracts of algae, seed extracts, fruit extracts, plant exudates, cellulose derivatives, gelling agents of animal origin, microorganism exudates, and synthetic gelling agents.

8. A composition according to claim 7, wherein said extracts of algae are selected from agar-agar, carrageenans and alginates; said seed extracts are selected from extracts of carob and extracts of guar; said fruit extract is pectin; said plant exudates are selected from gum arabic, gum tragacanth, karaya gum and ghatti gum; said cellulose derivative is carboxymethylcellulose; said gelling agents of animal origin are selected from gelatin and caseinates; said microorganism exudate is xanthan gum; and said synthetic gelling agents are selected from acrylic polymer derivatives and silicon derivatives.

9. A composition according to claim 7, wherein said at least one thickener is present in the aqueous phase in an amount of from 0.2 to 10% by weight relative to the aqueous phase.

10. A composition according to claim 1, wherein said at least one hydrophilic component represents from 4 to 40% by weight of the composition.

11. A composition according to claim 10, wherein said at least one hydrophilic component represents from 8 to 25% by weight of the composition.

12. A composition according to claim 1, further comprising at least one surfactant.

13. A composition according to claim 1, wherein said at least one volatile compound is selected from hydrocarbon oils, cyclic silicone oils, and linear silicone oils.

14. A composition according to claim 1, wherein said at least one volatile compound is selected from volatile cyclic silicones having from 3 to 8 silicon atoms, dimethylsiloxane/ methylalkylsiloxane cyclocopolymers, volatile linear silicones having from 2 to 9 silicon atoms, alkyltrisiloxanes, and volatile hydrocarbon oils.

15. A composition according to claim 14, wherein said volatile cyclic silicones have from 4 to 6 silicon atoms.

16. A composition according to claim 1, wherein said at least one volatile compound represents from 8 to 70% by weight relative to the total weight of the composition.

17. A composition according to claim 16, wherein said at least one volatile compound represents from 15 to 55% by weight relative to the total weight of the composition.

18. A composition according to claim 1, wherein said at least one pasty fatty compound has a viscosity at 40° C. of from 0.1 to 40 Pa.s and/or a melting point of from 25 to 70° C.

19. A composition according to claim 18, wherein said at least one pasty fatty compound has a viscosity at 40° C. of from 0.5 to 25 Pa.s and/or a melting point of from 25 to 55° C.

20. A composition according to claim 1, wherein said at least one pasty fatty compound is selected from hydrocarbon compounds.

21. A composition according to claim 1, wherein said at least one pasty fatty compound is a mixture selected from hydrocarbon compounds, silicone compounds and fluoro compounds, said hydrocarbon compounds being present in said mixture and being predominant.

22. A composition according to claim 1, wherein said at least one pasty fatty compound is selected from lanolins, lanolin derivatives, fatty esters, triglycerides, arachidyl propionate, polyvinyl laurate, cholesterol esters, pasty silicone fatty substances, thickened oils, and hydrogenated castor oil derivatives.

23. A composition according to claim 22, wherein said fatty esters have 20 to 45 carbon atoms, said triglycerides are hydrogenated plant oils and said pasty silicone fatty substances are alkyldimethicones.

24. A composition according to claim 22, wherein said thickened oil is thickened using at least one thickener selected from clays, aluminium and magnesium silicates, and polymers capable of thickening oils.

25. A composition according to claim 24, wherein said clays are selected from bentonites and hectorites, which are optionally modified with distearyldimethylammonium chloride or with stearyldimethylbenzylammonium chloride.

26. A composition according to claim 22, wherein said thickened oil is selected from
liquid paraffin, liquid petroleum jelly, perhydrosqualene, groundnut oil, sweet almond oil, beauty-leaf oil, palm oil, castor oil, avocado oil, jojoba oil, olive oil, cereal germ oil, fatty acid esters, alcohols, acetylglycerides, octanoates, decanoates and ricinoleates of alcohols and of polyalcohols, fatty acid triglycerides and glycerides;
polydimethylsiloxanes (PDMS) and alkyldimethicones;
silicones modified with aliphatic and/or aromatic groups, which may optionally contain fluorine, or with hydroxyl, thiol and/or amine functional groups; and
phenylated silicone oils of the following formula: 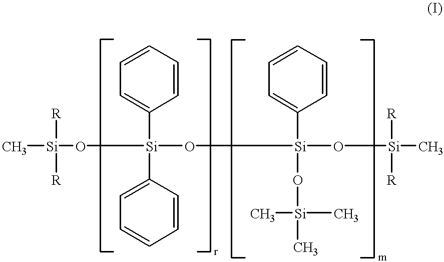
wherein
R is a C₁-C₃₀ alkyl radical, an aryl radical or an aralkyl radical,
n is an integer from 0 to 100,
m is an integer from 0 to 100, with the proviso that the sum m+n is from 1 to 100.

27. A composition according to claim 1, wherein said at least one pasty fatty compound is present in an amount of from 1 to 40% by weight relative to the weight of the composition.

28. A composition according to claim 27, wherein said at least one pasty fatty compound is present in an amount of from 8 to 35% by weight relative to the weight of the composition.

29. A composition according to claim 28, wherein said at least one pasty fatty compound is present in an amount of from 15 to 30% by weight relative to the weight of the composition.

30. A composition according to claim 1, further comprising at least one wax.

31. A composition according to claim 30, wherein said at least one wax is selected from microcrystalline waxes, paraffin, petrolatum, petroleum jelly, ozokerite, montan wax, beeswax, lanolin and derivatives thereof; candelilla wax, ouricurry wax, carnauba wax, Japan wax, cocoa butter, cork fibre wax, sugar cane wax, hydrogenated oils which are solid at 25° C., fatty esters and glycerides which are solid at 25° C.; polyethylene waxes, the waxes obtained by Fischer-Tropsch synthesis, silicone waxes, and fluoro waxes.

32. A composition according to claim 30, wherein said at least one wax is present in an amount of from 0.5 to 30% by weight relative to the total weight of the composition.

33. A composition according to claim 32, wherein said at least one wax is present in an amount of from 10 to 20% by weight relative to the total weight of the composition.

34. A composition according to claim 30, wherein said at least one wax is a hydrocarbon wax, said pasty fatty compound is selected from lanolins and lanolin derivatives, and said at least one volatile compound is selected from isoparaffins and cyclic silicones.

35. A composition according to claim 1, further comprising an oil of plant, mineral, animal, synthetic and/or silicone origin.

36. A composition according to claim 35, wherein said thickened oil is selected from
liquid paraffin, liquid petroleum jelly, perhydrosqualene, groundnut oil, sweet almond oil, beauty-leaf oil, palm oil, castor oil, avocado oil, jojoba oil, olive oil or cereal germ oil; fatty acid esters; alcohols; acetylglycerides; octanoates, decanoates or ricinoleates of alcohols or of polyalcohols; fatty acid triglycerides; glycerides;
polydimethylsiloxanes (PDMS) and alkyldimethicones;
silicones modified with aliphatic and/or aromatic groups, which may optionally contain fluorine, or with hydroxyl, thiol and/or amine functional groups;
phenylated silicone oils of the following formula: 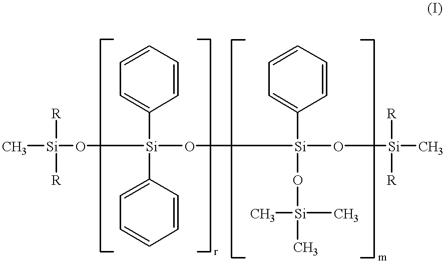
wherein
R is a C₁-C₃₀ alkyl radical, an aryl radical or an aralkyl radical,
n is an integer from 0 to 100,
m is an integer from 0 to 1 00, with the proviso that the sum m+n is from 1 to 100.

37. A composition according to claim 1, said composition comprising less than 20% by weight of at least one non-volatile hydrocarbon oil.

38. A composition according to claim 37, said composition comprising less than 15% by weight of at least one non-volatile hydrocarbon oil.

39. A composition according to claim 38, said composition comprising less than 5% by weight of at least one non-volatile hydrocarbon oil.

40. A composition according to claim 39, said composition comprising no non-volatile hydrocarbon oil.

41. A cosmetic, dermatological, hygiene and/or pharmaceutical composition comprising
at least one hydrophilic component and
a fatty phase which comprises at least one pasty fatty compound.

42. A composition according to claim 41, wherein said at least one pasty fatty compound is selected from lanolins and lanolin derivatives.

43. A composition according to claim 41, wherein said composition further comprises hydrocarbon waxes.

44. A composition according to claim 1, said composition further comprising a particulate phase comprising pigments and/or fillers and/or pearlescent agents.

45. A composition according to claim 1, said composition further comprising at least one agent selected from agents active against microorganisms, agents with anti-inflammatory or immunomodulatory activity, agents which antagonize neuromediators or which modulate the release of neuromediators, agents which modulate cell differentiation and/or proliferation and/or pigmentation and/or which regulate keratinization, agents active in the treatment and/or prevention of cheilitis, an antihistamine, and a cicatrizing agent.

46. A composition according to claim 1, wherein said composition is in the form of a stick or pencil, in the form of an oily liquid, which is optionally gelled, or in the form of a soft paste with a dynamic viscosity at 25° C. of from 3 to 35 Pa.s, measured with a Contraves TV rotary viscometer equipped with an “MS-r4” rotor at a frequency of 60 Hz.

47. A composition according to claim 1, wherein said composition is in the form of a care and/or make-up product for the skin, mucous membranes, semi-mucous membranes and the exoskeleton; a hygiene product; or a pharmaceutical product.

48. A composition according to claim 47, wherein said composition is in the form of a foundation, a blusher, an eyeshadow, a lipstick, a mascara, an eyeliner, a lipcare product, a fixing base for the lips, a self-tanning product or an antisun product.

49. A process for limiting, decreasing and/or eliminating the transfer and/or migration of a cosmetic, dermatological, hygiene and/or pharmaceutical composition comprising a hydrophilic component, said process comprising including in said composition at least one volatile compound and at least one pasty fatty compound.

50. A process according to claim 49, wherein said composition is a make-up or care composition for the skin, mucous membranes, semi-mucous membranes and/or the exoskeleton.

51. A process for limiting, decreasing and/or eliminating the transfer and/or migration of a cosmetic, dermatological, hygiene and/or pharmaceutical composition comprising a hydrophilic component, said process comprising including in said composition at least one pasty fatty compound.
